(19) United States
(12) Patent Application Publication
(10) Pub. No.: US 2010/0119617 A1
(43) Pub. Date: May 13, 2010

(54) MULTI-NUTRIENT MILK FORTIFIER

(75) Inventor: Deborah L. O'Connor, Toronto (CA)

Correspondence Address: BORDEN LADNER GERVAIS LLP;Anne Kinsman, WORLD EXCHANGE PLAZA, 100 QUEEN STREET SUITE 1100, OTTAWA, ON, K1P 1J9, CA

(73) Assignee: THE HOSPITAL FOR SICK CHILDREN, Toronto, ON (CA)

(21) Appl. No.: 12/593,442

(22) PCT Filed: Mar. 28, 2008
(86) PCT No.: PCT/CA2008/000582

§ 371 Date: Sep. 28, 2009

Related U.S. Application Data
(60) Provisional application No. 60/908,842, filed on Mar. 29, 2007.

(51) Int. Cl. A61K 33/42; A23J 1/00; A23C 9/00; A23L 1/30; A61K 38/16

(52) U.S. Cl. 424/602; 426/656; 426/580; 426/73; 514/12

(57) ABSTRACT

A multi-nutrient fortifier Total Randomized for supplementing breastfed infants is provided. The fortifier can be sterilized and can be formulated for delivery after hospital discharge. The fortifier is designed to supplement infants to meet the specific needs such as maintaining appropriate and safe levels of fat soluble vitamins and minerals, to incorporate vitamins normally requiring additional supplementation, such as vitamins A, D, E or iron for breastfed infants, to facilitate delivery of calcium and phosphorous salts. According to an embodiment of the invention, the fortifier can be in a concentrated liquid form, is sterile, and delivers greater than about 44% protein, by weight, and greater than about 17% protein, by weight, when analyzed in combination with breast milk. The fortifier can be used to improve growth and body composition of a breastfed infant post hospital discharge, such as a low birth weight infant, a post-surgical infant, or an ill term-born infant.

### CROSS-REFERENCE TO RELATED APPLICATIONS

The present application claims priority from U.S. Provisional Application 60/908,842, filed Mar. 29, 2007, the entirety of which is incorporated herein by reference.

### FIELD OF THE INVENTION

The present invention relates generally to multi-nutrient fortifiers. More particularly, the present invention relates to a multi-nutrient fortifier for breastfed infants.

### BACKGROUND OF THE INVENTION

Growth faltering and malnutrition among breastfed premature infants immediately following hospital discharge is a well described clinical concern (1-7). It is well accepted that the nutritional status and growth of premature infants influences their immediate chance of survival and frequency of rehospitalization, and a growing body of evidence suggests that it also impacts a number of other short- and long-term outcomes, including cognitive, language and motor development and bone mineralization (4, 6, 8-11). Physiological adaptations to accommodate a less than ideal metabolic environment (ie. over or under nutrition and formula feeding) may, in turn, program for an increased risk of cardiovascular disease, hypertension, insulin resistance, and diabetes mellitus in adulthood (9). The goal of the clinician, then, in nourishing premature infants should be to promote exclusive breastfeeding, minimize nutrient deficits, address them promptly, and avoid over nourishing infants once nutrient deficits are corrected. Despite a plethora of review articles and position statements from authoritative bodies encouraging breastfeeding, no randomized control trials had been conducted to ascertain whether multi-nutrient fortification of human milk after hospital would be beneficial (1-7).

Optimal nutrition during infancy is critical in the development of multiple organ systems. Less than an adequate supply of energy and essential nutrients at this time can have a profound impact on somatic growth, organ structure, and functional development (13). Malnutrition during infancy is known to impair brain development, reducing cell number, synaptic conductivity, neurotransmitter availability and function. In addition, infants that are malnourished grow poorly and are at increased risk of infection, poor bone mineralization, lethargy, and hospital re-admission. With time and improved nutritional status, many symptoms of malnutrition disappear but it is becoming increasingly apparent that some effects may be irreversible (14, 15).

Infants at greatest risk of malnutrition are those that are born prematurely or term-born infants that have elevated nutritional requirements due to surgery or significant morbidity. It is estimated that 6.7% of all live births in Canada are premature deliveries (16), translating into ˜23,015 premature births annually (17). In the United States it is estimated that 10.9% of all babies are born prematurely (16) which in turn translates into ˜461,451 births (US Census Data, CIA World Factbook, https://www.cia.gov/library/publications/the-world-factbook/geos/us.html). It is well accepted that LBW infants that grow well during the first couple years of life, and by extrapolation are not malnourished, have better cognitive and motor developmental outcomes and do better at school and in their social interactions, than those that grow poorly (10, 11, 19-24). For example, Hack et al demonstrated that VLBW infants (n=249) whose head size had not reached a normal size by 8 months CA had poorer verbal and performance IQ at 8 years of age than infants whose head size caught up by 8 months CA (21). In addition, infants with a sub-normal head size at 8 months CA had lower scores of receptive language, speech, reading, mathematics, and spelling and had a higher incidence of hyperactivity at 8 years of age.

Advances in neonatal care have resulted in increased survival rates among the smallest premature infants, frequently born at the end of the second and the beginning of the third trimester of pregnancy (16). As the vast majority of nutrient stores cross the placenta in the third trimester of pregnancy, as a general rule, the more premature an infant the greater is the risk of malnutrition. Further, the more premature an infant, the greater likelihood of a significant co-morbidity (e.g. chronic lung disease) which frequently elevates nutrient requirements (13, 25). Often the same co-morbidity may also limit the volume of feeding and/or route that nutrients are supplied, further complicating the provision of adequate nutrition (13). Organ immaturity and underdeveloped metabolic pathways secondary to prematurity, leads to an inability to maximize use of energy and nutrients provided. Fear of necrotizing enterocolitis (NEC), a common gastrointestinal emergency, often leads to an extremely cautious approach to the introduction and progression of feeding (6). A confirmed case of NEC may result in surgical intervention, a short gut, and long-term issues with nutrient absorption (6). Once fed orally, and often for some period of time after hospital discharge, many very premature babies demonstrate immature or uncoordinated sucking, swallowing, and breathing mechanisms which further limits nutrient intake (26).

Currently, the American Academy of Pediatrics (AAP) and the Canadian Pediatric Society (CPS) recommend that preterm infants should be provided with sufficient energy and nutrients to allow them to grow and accumulate lean body mass in their extra-uterine environment at a rate equal to that anticipated in utero (5, 12). Unfortunately, this is seldom achieved and many LBW infants, and particularly VLBW infants, leave hospital malnourished. Lemons et al illustrates this point in a report of ˜4,500 VLBW infants born at the 14 centres of the US Neonatal Research Network in 1995-1996 (27). Of these infants, 22% showed overt signs of under-nutrition at birth and 97% did so at 36 weeks' post-conceptional age, suggesting that infants go home from hospital at poorer nutritional status than they were at birth (27). These data are consistent with a number of others including 2 large cohorts of infants followed by Lucas in the United Kingdom, and Merko et al in Toronto, Ontario (28, 29). Embleton et al recently confirmed that preterm infants inevitably accumulate a significant nutrient deficit in the first few weeks of hospitalization that is not replaced when the recommended energy and nutrient intakes are fed. The nutrient deficit can be directly related to the degree of suboptimal growth and in the Embleton et al study, 45% of the growth was related to nutrient intake (30).

Despite these observations, there are few data on which to formulate evidence-based guidelines for feeding premature infants after hospital discharge (5, 12). While literally thousands of scientific studies exist to guide in-hospital feeding practices, until relatively recently issues of post-discharge nutrition have largely been ignored. The AAP acknowledges this is particularly problematic for the human milk fed infant after hospital discharge (5). Just recently the European Society for Gastroenterology, Hematology and Nutrition recommended routine multi-nutrient supplementation of human milk-fed infants with subnormal weight for postconceptional age as these babies are at elevated risk of growth failure after discharge (1). Given the data of Lemons and others summarized above, the recommendation implies that the vast majority of human milk-fed VLBW infants require nutrient supplementation after discharge. Interestingly, they extensively used published work of the instant inventor to justify this recommendation (31); somewhat alarming, however, is that there is not commercial product to implement it.

Infant formulas designed for premature infants after hospital discharge are widely available. As was nicely reviewed recently by Carlson, there is good evidence to suggest that the nutritional status of low birth weight infants as measured by weight, length or head circumference and/or biochemical indices (functional or static) is not corrected by hospital discharge (2). Most, but not all studies, with formula-fed infants, albeit few in number and limited in duration of follow-up, suggest that infant formula designed to contain higher concentrations of energy and nutrients (post-discharge formulas) will hasten the return of nutritional sufficiency early after hospital discharge compared to formulas designed for term-born infants (2, 8, 18, 32-38). In studies where improvements are found, it is the male and smaller infants that appear to achieve the greatest benefit of a nutrient-enriched formula. Infant formulas designed for premature infants after hospital discharge, commonly referred to as post-discharge formulas, are commercially available. Their energy and nutrient levels are between that of formulas designed for premature infants during their initial hospitalization and that of standard formulas designed for healthy term-born infants.

Breastfeeding is the optimal way to feed premature babies after hospital discharge; however no commercially available nutrient-supplement is available to address increased nutrient needs of many of these infants, and specifically VLBW infants. Breastfeeding is the gold standard and strongly preferred method of feeding healthy term-born and prematurely born infants (39-42). The scientific rationale for recommending breastfeeding as the preferred feeding choice stems from its documented benefit to infant nutrition, gastrointestinal function, host defense, neurodevelopment and psychological, economic, and environmental well-being (5, 42). In hospital, concentrated multi-nutrient fortifiers are added to human milk (˜1 gram fortifier per 25 ml milk) to produce nutrient intakes that will facilitate growth, tissue and nutrient accretion at rates equal to that anticipated in utero. Prior to hospital discharge, this process is usually discontinued, often abruptly, as an attempt is made to quickly transition a baby to feeding at the breast in preparation for hospital discharge. Depending on the philosophy of the nursery, human milk-fed low birth weight babies may be sent home exclusively at the breast, or fed a pre-determined number of bottles each day containing a discharge formula or human milk fortified with powdered infant formula. Anecdotally, breastfeeding mothers of these small babies describe feeling they are “thrown out on the street” with little support to address on-going feeding and growth issues after discharge. Weight gain is poor during the first weeks after discharge and some babies actually lose weight. Breastfeeding rates tend to be low after discharge and plummet rapidly. Mothers are often concerned that their baby is not obtaining enough milk or gaining weight and these reasons are frequently cited for discontinuing breastfeeding (43). Pediatricians in the community feel ill-equipped to formulate home-made recipes to add additional nutrients to mother's milk and introduction of nutrient-enriched proprietary formula is usually recommended.

Observational studies confirm the aforementioned anecdotal reports and they further suggest that the growth, lean body mass accretion, and bone mineralization of exclusively human milk-fed low birth weight infants lag considerably behind that of formula-fed infants premature infants after hospital discharge. This indicates that human milk-fed babies may be more malnourished at hospital discharge and have a slower rate of return to nutritionally adequacy (2, 19, 31, 47-52).

There is a need for a new multi-nutrient fortifier, preferably a post-discharge fortifier. As described above, there is no current mechanism or nutritional product on the market to facilitate adding a concentrated source of macro- and micro-nutrients to human milk after hospital discharge. The human milk fortifier used in the original study was formulated for in-hospital use and is not suitable for routine use after hospital discharge. Routine use without intense monitoring of nutrient intakes could result in unsafe levels of fat soluble vitamins and select minerals. As this product is not sterile there is a risk associated with its use in the much less controlled home-environment. In-hospital fortifiers are typically mixed with human milk by specially trained technicians using aseptic conditions in a laminar flow hood. As formulated the current in-hospital fortifier requires separate vitamin/mineral drops to meet the unique needs of the post-discharge infant (e.g. additional vitamin D and iron) and ideally these nutrients should be incorporated into the fortifier. For other nutrients (e.g. vitamin A), in combination with currently commercially available and prescribed vitamin and mineral drops, unsafe levels could be consumed. Finally, its formulation doesn't facilitate maximum delivery of nutrients (e.g. fall-out of calcium and phosphorus salts) and doesn't reflect the current understanding of ingredient selection to facilitate ease of use, feeding tolerance and reduced allergenicity.

Few options are currently available to parents after hospital discharge. Nutritional products designed for premature and/or sick infants are typically manufactured by companies that make infant formula. The formula industry has made a significant contribution to the advancement of pediatric research. However, of the few products available for the breast-fed infant, all are designed for in-hospital use by the premature infant, none are designed for use after hospital discharge or for the sick exclusively breastfed term-born infant. In addition, current in-hospital fortifiers can be prohibitively expensive (for example, as much as $60 per ounce for certain fortifiers that use concentrated human milk). The post-discharge fortifier in accordance with the present invention should promote increased breastfeeding after hospital discharge.

There is an interest in North America as well as elsewhere in the world in products that promote the well-being of infants and children. Interest comes from a variety of sectors such as industry, hospitals, parents, and breast-feeding advocates. There is great demand and need for a post-discharge multi-nutrient fortifier to supplement the nutrients delivered in breast milk.

There is also interest in multi-nutrient fortifiers for term-born sick infants, for example, a fortifier to be added to breast milk for babies post-cardiac surgical complications (ie. chylothorax), gastro-intestinal surgery etc.

There is no commercially available concentrated multi-nutrient fortifier available that can safely, and affordably be routinely added to or consumed in conjunction with human milk after hospital discharge. There is a need for such a fortifier, directed to low birth weight infants, and also post-surgical or ill term-born infants. It is, therefore, desirable to provide a multi-nutrient fortifier that is specifically formulated to meet the needs of breastfed low birth weight infants, and which may be particularly advantageous for use after hospital discharge.

### SUMMARY OF THE INVENTION

It is an object of the present invention to obviate or mitigate at least one disadvantage of previous human milk fortifiers.

In a first aspect, the present invention provides a multi-nutrient fortifier for supplementing a breastfed infant. The fortifier can be sterilized; it can also be formulated for delivery after hospital discharge. In one embodiment, the fortifier can be in a concentrated liquid form. The infants are typically low birth weight (LBW) infants, post-surgical or ill term-born infants. Typically, the fortifier has greater than about 44% protein by weight of the energy-contributing ingredients therein; when mixed with human milk, the formulation (fortifier+human milk) is typically about 17% protein by weight of the energy-contributing ingredients therein.

In one particular embodiment, the formulation comprises, per 100 mL, the following nutrients: a) energy: 81±about 20% kcal; b) 2.7 g±about 20% hydrolyzed whey protein; c)4.4 g±about 20% of fat; d) 8.9 g±about 20% of carbohydrate; e) 138.8 mg±about 20% of calcium; f) 69.4 mg±about 20% of phosphorus; g) 9.7 mg±about 20% of magnesium; h) 37 mg±about 20% of sodium; i) 110 mg±about 20% of potassium; j) 55 mg±about 20% of chloride; k) 1.2 mg±about 20% of zinc; I) 0.08 mg±about 20% of copper; m) 2.0 mg±about 20% of iron; n) 4 μg±about 20% of selenium; o) 100 μg±about 20% of Vitamin A; p) 150 IU±about 20% of Vitamin D; q) 1.5 mg±about 20% of Vitamin E; r) 30 μg±about 20% of folic acid; s) 1.0 μg±about 20% of manganese; t) 0.07 mg±about 20% of thiamin; u) 0.1 mg±about 20% of riboflavin; v) 0.8 mg±about 20% of niacin; w) 1.7 μg±about 20% of biotin; x) 0.0405 mg±about 20% of Vitamin B6; y) 0.14±about 20% of Vitamin B12; z) 0.7 μg±about 20% of Vitamin K; aa) 27.5 mg±about 20% of Vitamin C; bb) 0.55 mg±about 20% of pantothenic acid; and cc) 22.5 mg±about 20% of choline; when provided together with human breast milk. In addition to these nutrients, other nutrients may also be added, such as any nutritionally appropriate vitamin or mineral.

The multi-nutrient fortifier in accordance with the present invention is particularly suitable for supplementing breastfed infants, such as LBW infants, post-surgical infants or ill term-born infants, and formulated to overcome at least one of the following characteristics of human breast milk fortified with a conventional human milk fortifier:

(1) at the volumes of milk ingested, infants may consume unsafe levels of fat soluble vitamins and select minerals with conventional fortifiers;

(2) conventional fortifiers are not sterile which makes feeding it to malnourished and immuno-compromised infants in the less controlled home-environment a risk;

(3) even when conventional fortifiers are used, infants still require separate vitamin/mineral drops to meet the unique needs of the post-discharge infant (e.g. additional vitamin D and iron) but unfortunately, these nutrients are not incorporated into conventional fortifiers;

(4) the formulation of a conventional fortifier doesn't facilitate maximum delivery of nutrients (e.g. fall-out of calcium and phosphorus salts); or

(5) a conventional fortifier doesn't reflect the current understanding of ingredient selection to facilitate ease of use, feeding tolerance and reduced allergenicity.

Advantageously, the fortifier has greater than about 44% by weight protein of the energy-contributing ingredients therein; when mixed with human milk, the formulation is typically about 17% by weight protein of the energy-contributing ingredients therein.

Surprisingly, the fortifier in accordance with the present invention has been shown to be an effective supplement to human breast milk for a breastfed infant for improving growth and body composition of the infant.

The fortifier can be provided as a powder, paste, concentrated liquid, or in another acceptable form. One advantage of the embodiment in which the fortifier is provided as a concentrated liquid is that direct mixing with breast milk need not be conducted, if for example, a supplemental feeder may be used to deliver the concentrated liquid while the infant is at the breast.

Sterilization of the fortifier can be particularly advantageous in providing a safer formulation to the infant. Sterilization of the fortifier can be achieved using any appropriate method in the art. For example, sterilization of a liquid form of the fortifier of the present invention may be accomplished through filtration or by other means such as radiation and/or heat or pressure treatments.

In accordance with another aspect of the present invention there is provided a method of improving growth and body composition of a breastfed infant comprising: supplementing human breast milk with a multi-nutrient fortifier as described herein to produce a supplemented breast milk formulation; and administering to the infant the supplemented breast milk formulation, thereby improving growth and body composition of the infant. More particularly, the breastfed infant can be a low birth weight infant, post-surgical or ill term-born infant. Growth and body composition have been shown to be improved in the infant, particularly post hospital discharge.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures.

FIG. 1 shows weight, length and head circumference measurements until 6 months CA of exclusively human milk-fed infants (n=39) sent home (Study Day 1) fed human milk alone (−) or with ˜½ of the human milk fed mixed with a multinutrient fortifier (−−) for 12 weeks. Feeding groups differed (weight, P=0.1; length, P=0.02; head circumference, P=0.0008).

FIG. 2 shows the visual acuity at 4 and 6 months CA of exclusively human milk-fed infants (n=39) sent home fed human milk alone (−) or with ˜½ of the human milk fed mixed with a multinutrient fortifier (−) for 12 weeks following hospital discharge. Data are presented as mean spatial frequency (cycles/degree, cpd)±SD octaves. Feeding groups differed (P=0.02).

FIG. 3 is a flow chart showing overall study design.

### DETAILED DESCRIPTION

Generally, the present invention provides a multi-nutrient fortifier. More particularly, the present invention provides a multi-nutrient fortifier for supplementing a breastfed infant. The fortifier can be sterilized; it can also be formulated for delivery after hospital discharge. The infant can be a low birth weight infant (LBW), a post-surgical infant, or an ill term-born infant.

In one embodiment, the formulation is in a concentrated liquid form. Typically, the fortifier has greater than about 44% or more protein, by weight of the energy-contributing ingredients therein; when mixed with human milk, the formulation (fortifier+human milk) is typically about 17% by weight protein of the energy-contributing ingredients therein.

Conventional multi-nutrient fortifiers, such as those known in the art, may not be suitable for routine use after hospital discharge. Typically, there are several factors contributing to this unsuitability. For example, at the volumes of milk ingested after discharge, infants may consume unsafe levels of fat soluble vitamins and select minerals. Second, conventional multi-nutrient fortifiers are usually not sterile, which can compromise the health of malnourished and immuno-compromised infants when feeding them in the less controlled home-environment. In addition, infants still require separate vitamin/mineral drops to meet the unique needs of the post-discharge infant (e.g. additional vitamin D and iron) and ideally these nutrients are not typically incorporated into the fortifier. Standard formulations do not ideally facilitate maximum delivery of nutrients (e.g. fall-out of calcium and phosphorus salts). The multi-nutrient fortifier of the present invention seeks to incorporate appropriate ingredients to facilitate ease of use, feeding tolerance and reduced allergenicity.

Ideally, the multi-nutrient fortifier in accordance with the present invention is particularly suitable for supplementing breastfed infants, such as LBW, post-surgical or ill term-born infants, and formulated to overcome at least one of the following characteristics of human breast milk fortified with a conventional human milk fortifier: (1) at the volumes of milk ingested, infants may consume unsafe levels of fat soluble vitamins and select minerals with conventional fortifiers; (2) conventional fortifiers are not sterile which makes feeding it to malnourished and immuno-compromised infants in the less controlled home-environment a risk; (3) even when conventional fortifiers are used, infants still require separate vitamin/mineral drops to meet the unique needs of the post-discharge infant (eg. additional vitamin D and iron) but unfortunately, these nutrients are not incorporated into conventional fortifiers; (4) the formulation of a conventional fortifier doesn't facilitate maximum delivery of nutrients (eg. fall-out of calcium and phosphorus salts); or (5) a conventional fortifier doesn't reflect the current understanding of ingredient selection to facilitate ease of use, feeding tolerance and reduced allergenicity.

According to one embodiment of the invention, the post-discharge fortifier can be a liquid and/or in a concentrated form. However, powdered formulations (or any other suitable formulations) having the appropriate characteristics also fall within the scope of the invention. A concentrated liquid fortifier has an advantage of reducing and/or eliminating the need to have mothers pump milk to add the fortifier to and would increase the number of feedings that could take place directly at the breast.

Sterilization of a fortifier in accordance with the present invention, such as a liquid fortifier for example, may be accomplished through filtration, or by other means such as radiation and/or heat or pressure treatments. The fortifier can be manufactured using any suitable means in the art.

As used herein, “breastfed” can mean that the infant can receive human milk by directly suckling at the mother's breast, by bottle, or via a feeding tube.

In one particular embodiment, the formulation (fortifier+human milk) comprises, per 100 mL total volume, the following nutrients: a) energy: 81± about 20% kcal; b) 2.7 g±about 20% hydrolyzed whey protein; c) 4.4 g±about 20% of fat; d) 8.9 g±about 20% of carbohydrate; e) 138.8 mg±about 20% of calcium; f) 69.4 mg±about 20% of phosphorus; g) 9.7 mg±about 20% of magnesium; h) 37 mg±about 20% of sodium; i) 110 mg±about 20% of potassium; j) 55 mg±about 20% of chloride; k) 1.2 mg±about 20% of zinc; I) 0.08 mg±about 20% of copper; m) 2.0 mg±about 20% of iron; n) 4 μg±about 20% of selenium; o) 100 μg±about 20% of Vitamin A; p) 150 IU±about 20% of Vitamin D; q) 1.5 mg±about 20% of Vitamin E; r) 30 μg±about 20% of folic acid; s) 1.0 μg±about 20% of manganese; t)0.07 mg±about 20% of thiamin; u) 0.1 mg±about 20% of riboflavin; v) 0.8 mg±about 20% of niacin; w) 1.7 μg±about 20% of biotin; x) 0.0405 mg±about 20% of Vitamin B6; y) 0.14±about 20% of Vitamin B12; z) 0.7 μg±about 20% of Vitamin K; aa) 27.5 mg±about 20% of Vitamin C; bb) 0.55 mg±about 20% of pantothenic acid; and cc) 22.5 mg±about 20% of choline; when provided together with human breast milk. In addition to these nutrients, other nutrients may also be added, such as any nutritionally appropriate vitamin or mineral.

In accordance with another aspect of the present invention, there is provided a method of improving growth and body composition of a breastfed infant comprising: supplementing human breast milk with the multi-nutrient fortifier as described herein to produce a supplemented breast milk formulation; and administering to the infant the supplemented breast milk formulation, thereby improving growth and body composition of the infant. More particularly, the breastfed infant can be a low birth weight infant, post-surgical or ill term-born infant. Growth and body composition have been shown to be improved in the infant, particularly post hospital discharge. As mentioned herein, the multi-nutrient fortifier can be in a concentrated liquid or other suitable form. Ideally, the fortifier has greater than about 44% by weight protein of the energy-contributing ingredients therein; when mixed with human milk, the formulation is typically about 17% by weight protein of the energy-contributing ingredients therein.

As used herein, improving growth and body composition refers to improving one or more aspects of the infant (e.g., low birth weight infant (LBW), a post-surgical infant, or an ill term-born infant) post hospital discharge. These aspects can include but not limited to, improving visual development, general development level, weight, length, head circumference, or cognitive/language/motor/social-emotional/adaptive behaviour. Ideally, the improvement in growth and development can refer to improving growth with a proportional increase in bone and lean body mass. It would be understood that other developmental aspects may be considered within the context of the present invention.

### Example

As mentioned above, studies have indicated that human milk-fed babies may be more malnourished at hospital discharge and have a slower rate of return to nutritionally adequacy (2, 19, 31, 47-52). However, observational studies can often be confounded by other differences between groups.

In hospital settings, the addition of multi-nutrient fortifiers to human milk in order to improve the growth and development of low birth weight (LBW, <1800 grams), and particularly very LBW (VLBW, <1500 grams) infants is the current standard of care (5, 6, 12). While malnutrition among breastfed premature infants immediately following hospital discharge is a well described clinical concern, up until now there has been little research evidence to suggest a benefit of a proactive approach to nutrition post-discharge (1-7) Nonetheless the level of concern has risen with the increased survival of smaller premature infants, their proportionally higher nutrient-deficits and with greater emphasis on rapid hospital discharge. Today, more premature infants that ever are going home at risk of malnutrition (3, 6, 7, 12).

To address this, and funded by a Strategic Grant from the Institute of Musculoskeletal Health and Arthritis, of the Canadian Institute of Health Research, a randomized controlled study was completed in which 39 exclusively human milk-fed infants were assigned at hospital discharge to receive either their mother's milk alone (control, current clinical practice) or ˜½ of their mother's milk each day for 12 weeks with a multi-nutrient fortifier.

### Ethics

The study protocol was approved and annually reviewed by the Human Ethics

Committee at the Hospital for Sick Children and at each recruiting hospital. The research was conducted according to the policies and procedures of each institution and the Canadian Tri-Council policy statement on ethical conduct of research involving human subjects (30). This study was registered with the U.S. National Library of Medicine found at http://www.ClinicalTrials.gov (Registration #NCT00413985).

As described in detail elsewhere, the sample size for this pilot study was determined a priori to enable detection of a 1 standard deviation (SD) difference in the mean weight of infants in the two treatment groups at the end of feeding intervention with 80% power at an α-level of 0.05 (69). Based on a review of the literature at the time, that a sample size of 34 infants was sufficient to detect at least a 0.5 octave difference in visual acuity (31). This sample size was not identified as being sufficient to detect hypothesized differences (−0.25 SD) on the MDI or PDI of the BSIDII. The BSID-II was included in the study to assess if visual acuity and contrast sensitivity scores at 4 and 6 mo CA were associated with a later measure of global development, and to determine the appropriate sample size for inclusion of the BSID in larger future studies (32).

All data were analyzed using the Statistical Analysis System (SAS) for Windows version 9.1 (SAS Institute, Cary, N.C.). Statistical tests were two-tailed and used an α-level of 0.05. The normality of all data distributions were verified (PROC UNIVARIATE), and data were transformed to produce a normal distribution where possible. Continuous variables measured at >1 time point were analyzed using a mixed repeated-measures analysis of variance (PROC MIXED). Tukey's honestly significant difference procedure was used for pair-wise comparisons if a group by time interaction P≦0.15 was found. Correlations between: (1) change in anthropometric z-scores during the feeding intervention and visual outcomes at 4 and 6 mo CA; and (2) visual outcomes at 4 and 6 mo CA and the MDI, PDI and Behaviour Rating Scale at 18 mo CA were assessed using Pearson product moment correlations (r) if both variables passed normality tests or by Spearman rank correlations (rₛ) if one or both variables failed normality tests.

Infants that were born small for gestational age (SGA) were included in the study because they tend to represent a sizeable proportion of the preterm population. Since SGA infants often exhibit different growth patterns compared to their appropriate for gestational age (AGA) counterparts, we have presented the z-scores in Table 4 for AGA infants only. All statistical analyses were performed with and without SGA infants in the dataset to ensure that their inclusion did not influence study findings.

Briefly, 39 human milk-fed infants, and their mothers were enrolled from level II and level III neonatal intensive care nurseries located in the greater Toronto area (GTA). Infants born <33 weeks gestation, weighing between 750-1800 g, and who received ≧80% energy from human milk (at breast, expressed, unfortified, fortified) at the time of hospital discharge were eligible to participate. Study families had to agree to predominately feed their infants human milk after hospital discharge, and add extra nutrients to a predetermined volume using a powdered multi-nutrient fortifier, if so randomized. The fortifier used in the study was Similac Human Milk Fortifier. It is a fortifier that was developed for “in-hospital use” and is not suitable for routine use after hospital discharge unless there is very close monitoring of individual nutrient intakes which seldom occurs in the community setting.

Exclusion criteria included: serious congenital or chromosomal anomalies that could affect growth, grade ≧III periventricular or intraventricular hemorrhage, oral steroid treatment within 2 weeks of randomization, severe asphyxia, maternal substance abuse, and inability of the mother to verbally communicate in English. Infants were also excluded if the clinical team anticipated that post-discharge any single feeding would need to be energy and/or nutrient-enriched to ≧24 kcal/fl oz or if >50% of daily feeds needed to have energy and/or nutrients added.

Prevalence of serious adverse events and the prevalence and duration of hospital re-admissions of infants between Study Day 1 and 12 wk following initial hospital discharge and between Study Day 1 and each infant's 12 months CA birth date was examined.

The day before hospital discharge (Study Day 1), infants were randomized to either a control or intervention group using a computer-generated randomization schedule, stratified for gender and birth weight (≦1250 g, >1250 g). Families in both feeding groups were provided with intensive lactation support after hospital discharge which included unlimited use of an electric breast-pump (Purely Yours Breast Pump, Ameda, Mississauga, ON) and an experienced lactation consultant.

Families in the control group were sent home on unfortified human milk and vitamin drops consisting of vitamin A (1500 IU), D (400 IU) and C (30 mg), and iron (15 mg) drops as is routine clinical practice in the GTA. In the event that an infant in the control group demonstrated poor growth, as defined by pre-established algorithm (69), the child's pediatrician was advised. In these instances, it was at the discretion of the child's pediatrician how nutrient-enrichment was to be accomplished for infants in the control group; generally, a powdered post-discharge formula (eg. Similac Neosure) was added to human milk.

Families of infants assigned to the intervention were asked for 12 weeks following discharge to mix ˜½ of the human milk fed each day with extra energy and nutrients. The study co-ordinator provided caregivers with individualized recipes on how to mix a pre-determined volume of human milk (fresh or thawed) each day (150 ml×infant weight [kg]÷2) with a powdered in-hospital fortifier (Similac Human Milk Fortifier, Abbott Nutrition, Montreal, QC, 4 single-use packets [0.9 g each] per 100 mL expressed human milk). Recipes were calculated at hospital discharge and again at 4 and 8 weeks post-discharge to accommodate changes in infant weight. The remainder of feedings were to be provided as unfortified milk (from breast or expressed). In combination with unfortified human milk feedings, the average daily energy and nutrient density provided to infants in the intervention group was predicted to be similar to those of infants fed commercially available nutrient-enriched formula designed for post-discharge feeding of premature infants (eg. 22 kcal/fl oz and 18 g/L protein) (6). The energy and nutrient profile of the nutrient-enriched human milk fed to infants in the intervention is published in detail elsewhere (69).

The nutrient composition of these feedings is provided in Table 1. This study systematically examined whether extending in-hospital multi-nutrient fortification of human milk for a finite period of time after the babies go home is feasible, safe and improves short-term growth and development of low birth weight infants.

TABLE 1
Approximate Energy and Select Nutrient Composition
of Mature Preterm Human Milk (HM) and Mature
Preterm HM Supplemented with Similac ™ Human
Milk Fortifier per 100 mL

| | | B. HM with HM Fortifier |
|---|---|---|
| | A. Mature HM * | (4 pkts in 100 ml) |
| Nutrients | | |
| Energy (kcal) | 65 | 77 |
| Protein (g) | 1.2 | 2.2 |
| Fat (g) | 4.0 | 4.2 |
| Carbohydrate (g) | 7.4 | 9.0 |
| Minerals | | |
| Calcium (mg) | 26.4 | 139.6 |
| Phosphorus (mg) | 12.4 | 77.3 |
| Magnesium (mg) | 3.4 | 10.1 |
| Sodium (mg) | 16.0 | 30.2 |
| Potassium (mg) | 53.4 | 113.3 |
| Chloride (mg) | 42.6 | 78.4 |
| Zinc (mg) | 0.27 | 1.2 |
| Copper (mg) | 0.06 | 0.22 |
| Iron (mg) | 0.10 | 0.44 |
| Choline (mg) | 18.5 | 19.8 |
| Selenium (μg) | 1.8 | 2.2 |
| Vitamins | | |
| Vitamin A (ug) | 48.5 | 650.9 |
| Vitamin D (IU) | 26.0 | 142.2 |
| Vitamin E (mg) | | |
| (dl-α tocopherol) | 0.5 | 2.0 |
| Folic Acid (μg) | 8.5 | 30.7 |

* Milk nutrient composition values were obtained from: Institute of Medicine, Dietary Reference Intake Series (1997-2004). The series can be assessed at: http://www.iom.edu/CMS/3788/4574/45127.aspx. Where possible, milk composition values were used from studies that reported data on mature milk samples collected from mothers who delivered prematurely.

In order to avoid potentially high intakes of fat soluble vitamins, infants in the intervention group were provided with vitamin drops containing only 200 IU of Vitamin D (ie. ½ manufacturer recommended dose of D-Vi-Sol [Mead Johnson Nutritionals, Ottawa, Canada]). Vitamin A and C drops were not provided. They also received a daily iron supplement of 15 mg/d to address the low iron content of the human milk fortifier used in this study. In some countries, such as the US, vitamin D by itself is not readily available, further making it difficult to use in-hospital fortifiers after hospital discharge.

Nutrient Intakes, Duration of Breastfeeding and Feeding Protocol Compliance.

Percent of milk feeds as human milk (unfortified or fortified) at 4, 8 12 weeks post-discharge and at monthly intervals up to each infant's 12 months CA birth date starting at 3 months CA was examined. These data are also be compared to breasting mother/infant dyads that declined participation in the larger study. Volume of human milk, fortified human milk and formula consumed at 4 and 12 week post-discharge and estimated energy and nutrient intakes at 4 and 12 weeks post-discharge are determined.

The following formulation is an exemplary formulation according to the invention. Values shown in Table 6 represent values in human milk alone, versus the human milk plus the fortifier according to the invention. Additionally, two control products are shown, NeoSure™ and EnfaCare™, for comparative purposes. The two control products are infant formulas designed for use by preterm infants after hospital discharge so as not to confuse with a human milk fortifier. Table 7 provides more detailed information about the human milk alone versus the fortified composition plus human milk.

In one embodiment of the present invention, the post-discharge fortifier can be a liquid and/or a concentrate. This would reduce and/or eliminate the need to have mothers pump milk and hence would increase the number of feedings that could take place directly at the breast.

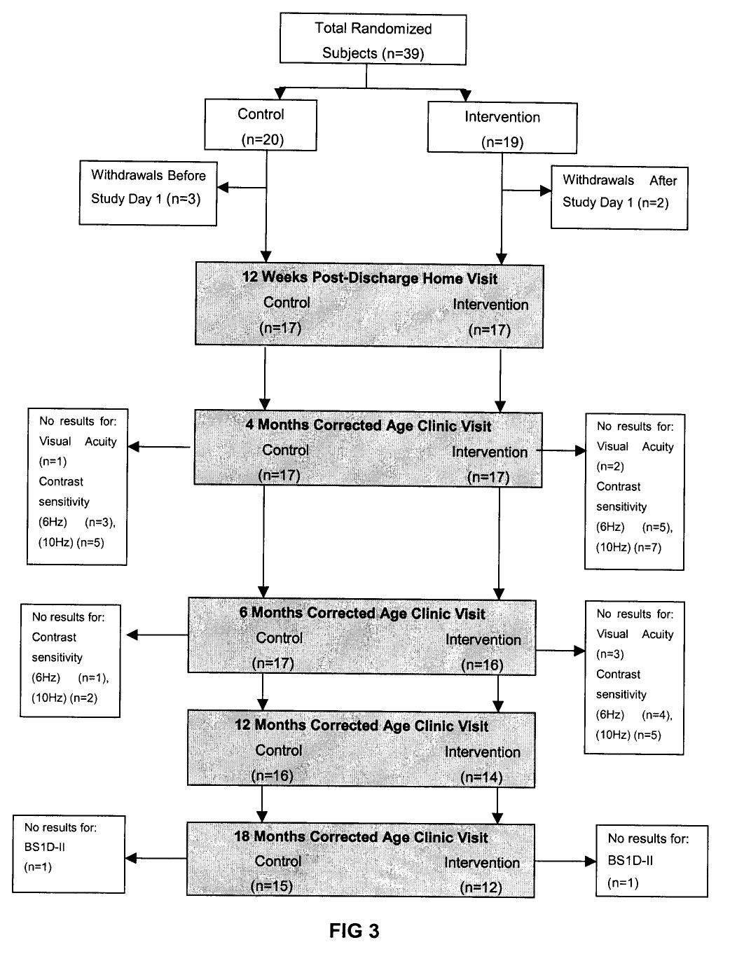

The overall study design is illustrated in FIG. 3.

Thirty-nine infants were randomized to either the control group (n=20) or the intervention group (n=19) (Table 2).

TABLE 2
Study characteristics of the control and
intervention groups upon enrollment¹

| | Control | Intervention |
|---|---|---|
| Characteristics | n = 20 | n = 19 |
| Birth Weight (g) | 1322 ± 332 | 1253 ± 242 |
| Gestational age at birth² (wk) | 29.8 ± 1.7 | 28.9 ± 1.2 |
| Sex³, male [n (%)] | 11 (47) | 14 (42) |
| Postconceptional age at study day 1 (wk) | 38.4 ± 2.4 | 37.8 ± 3.3 |
| Chronic lung desease⁴ [n (%)] | 5 (25) | 8 (42) |
| Maternal age (y) | 33 ± 4 | 34 ± 5 |
| Paternal age (y) | 35 ± 4 | 35 ± 7 |
| Retinopathy of prematurity ≧ Stage 3 | 0 | 0 |

¹Values presented are unadjusted means ± SD (number of subjects) unless otherwise indicated. Difference between feeding groups for continuous variables were assessed by 2-sided t test and for categorical variables using x² analyses
²P = 0.06
³P = 0.07
⁴Chronic lung disease was defined as the need for supplemental oxygen beyond 1 month chronological age or 36 weeks postconception

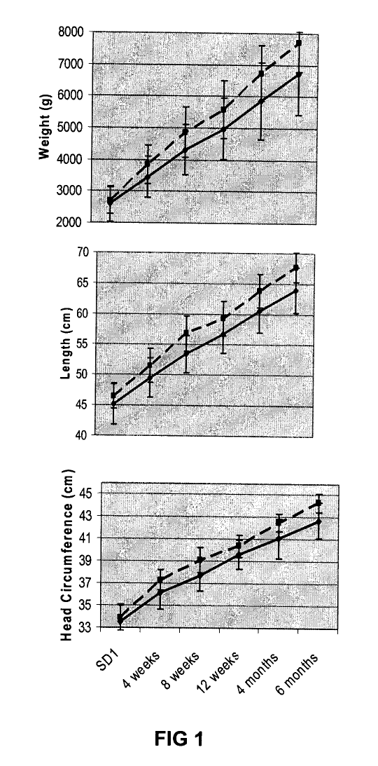

Of these infants, 34 completed the 12-week post-discharge intervention phase and attended the 4 mo CA clinic visit (17 per group). Two infants in the control group were fed human milk containing a powdered post-discharge formula during the feeding intervention phase to address poor growth but were included in all statistical analyses as randomized. Thirty-four and 33 infants attended their 4 and 6 mo CA clinic visit, respectively. The results for one infant in the intervention group were not included in the statistical analyses as this baby was being queried for hydrocephalus. As summarized in FIG. 1, results for some VEP testing conditions were not obtained; usually because the infant no longer was interested in the task or could not be calmed. Where possible, infants were brought in for a second testing session. Thirty infants and 29 infants remained in the study until the 12 and 18 month CA, respectively. Results for one infant in the control group were not used as the BSID-III was used instead of the BSID-II.

Most baseline infant and family demographics, including infant weight at Study Day 1, did not differ statistically between feeding groups (Table 2). There was a trend toward older gestational age at birth in the control (29.8+1.7 weeks) versus intervention (28.9+1.2 weeks) group (p=0.06), and fewer male infants in the control (11 of 20) versus intervention (14 of 19) group (p=0.07). The potential impact of these trends on outcomes was addressed by including the randomization strata (birth weight and sex), as planned, in all statistical analyses. There were no differences between the groups with regard to the in-hospital measures collected (eg. cases of systemic infection and necrotizing enterocolitis, days on parenteral nutrition, days to full enteral feeding).

During the feeding intervention there was no difference between feeding groups in the volume of human milk ingested. As illustrated in FIG. 1, added nutrients resulted in early post-discharge gains in length, and head circumference measurements that were sustained until at least 6 months CA. Likewise, infants in the +nutrients group (75.4+15.0 and 185.8+29.1) had a greater mean bone mineral content (grams) than infants in the control group (54.8+25.8 and 156.0+39.8) at 4 months and 12 mo CA, respectively (P=0.02).

Visual Development

A non-invasive sweep visual evoked potential (VEP) procedure, administered by a single tester (KW), unaware of feeding assignments, was used to assess the visual acuity and contrast sensitivity of infants at 4- and 6-mo CA (±7 d) (23, 24). Simply, visual acuity is the smallest detail a person can perceive, while contrast sensitivity is the lowest amount of contrast one can detect. Visual acuity was always assessed first at each testing session. For the VEP procedure, infants were seated on a parent's lap 100 cm from a 17 inch (43 cm) monitor (Power Mac G3 Pegasus M T, Apple Computer, Cupertino, Calif.) which displayed the visual stimulus to the child with mean space-average luminance of 80 cd/m². High contrast (80%) black and white horizontal square-wave gratings were displayed on the monitor to test visual acuity. Patterns were contrast reversed at 6.0 Hz and swept from 2.0 to 15.0 cycles/degree. To assess contrast sensitivity (at 6.0 and 10.0 Hz), black and white horizontal square gratings were swept logarithmically from 0.5% to 20% contrast at a fixed spatial frequency of 0.5 cycles/degree. Five sweeps each were performed for visual acuity and contrast sensitivity at 6 and 10 Hz over a 10 second intervals.

Five gold cup electrodes (Grass-Telefactor, West Warwick, R I) were positioned on the scalp at O_{z}, O₁, O₂, P_{Z}, and C_{Z}, according to the International 10-20 Electrode Placement System to capture the infant's brain response evoked by the visual stimuli. A differential amplifier (model 12 Data Acquisition System 12C-8-32; Grass Telefactor) was used to amplify the cortical response. The amplitude and phase of the evoked response were calculated by the recursive least squares (RLS) method from the second harmonic response. Visual acuity and contrast sensitivity thresholds were determined by Power Diva software (The Smith-Kettlewell Eye Research Institute, San Francisco, Calif.) designed to produce linear extrapolation of the evoked response to zero amplitude based on a fixed signal-to-noise ratio (≧3:1), phase and the T²-circ statistic (P<0.05).

It is known that ocular abnormalities and refractive error can influence visual acuity and contrast sensitivity. Therefore, each infant's medical chart was reviewed at hospital discharge to ascertain whether, or not, they had retinopathy of prematurity ≧Grade III. Further, opthalmologic assessments were collected for infants that had an eye exam between 6 and 12 mo CA. If infants did not have an eye exam as is recommended practice, the study coordinator helped to arrange this examination during a clinic visit. The opthalmologic assessment included an evaluation of refractive error and the impression of the fundus.

At 4 and 6 mo CA, infants in the intervention group demonstrated increased visual acuity compared to the control group (P=0.02) (Table 3). While there was no significant difference in mean contrast sensitivity between infants in the intervention and control groups at the higher temporal frequency (10 Hz), there was an interaction between feeding group and birth weight strata at the lower temporal frequency (6 Hz). Infants born >1250 g in the intervention group demonstrated greater contrast sensitivity compared to control infants (P=0.04). Removal of the SGA infants from all of the aforementioned statistical analyses did not alter the results for visual acuity or contrast sensitivity.

TABLE 3
Visual acuity and contrast sensitivity of
predominantly human milk-fed preterm infants¹

| | | Visual acuity² | Contrast | Contrast |
|---|---|---|---|---|
| Age and | | (cycles/degree ± | sensitivity³ | sensitivity⁴ |
| feeding group | | octaves) | (log, 6 Hz) | (log, 10 Hz) |
| 4 mo | Control | 6.9 ± 0.2 (16) | 1.8 ± 0.3 (14) | 1.7 ± 0.2 (12) |
| | Intervention | 7.8 ± 0.4 (16) | 1.8 ± 0.3 (13) | 1.8 ± 0.3 (10) |
| 6 mo | Control | 8.2 ± 0.4 (17) | 1.8 ± 0.3 (16) | 1.9 ± 0.2 (15) |
| | Intervention | 9.7 ± 0.2 (14) | 1.9 ± 0.2 (13) | 2.0 ± 0.3 (11) |

¹Values are the unadjusted means ± SD of all study infants tested. Differences between feeding groups were assessed by repeated measures analysis of variance controlling for gender and birth weight stratum (≦1250 g, >1250 g)
²Feeding Group Main Effect; P = 0.02; Time P = 0.0013
³Feeding group × birth weight stratum interaction (P = 0.07). Among infants ≧1250 g, Intervention > Control (P = 0.04)
⁴Feeding Group Main Effect; P = 0.11; Time P = 0.01

Review of opthalmologic assessments revealed no any abnormalities of the eye in any infant. There was no difference in mean refractive error between groups ([mean spherical equivalent±SD] control: 1.5±1.3, intervention: 1.2±1.0, P=0.63). No infant in the study had retinopathy of prematurity ≧Stage 3 at hospital discharge.

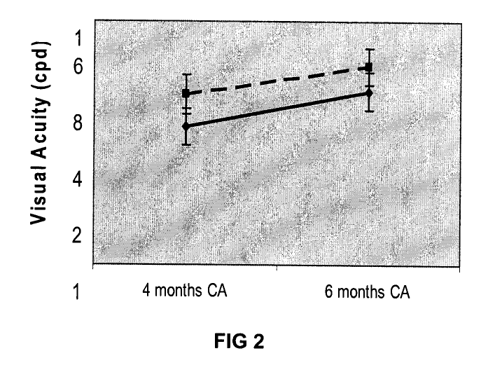

FIG. 2 shows that using a sweep visual evoked potential procedure to measure visual development at 4 and 6 months CA, infants in the nutrient-enriched group had improved visual acuity at 4 and 6 months CA (P=0.02). The latter observations of improved visual acuity among infants in the nutrient-enriched group represents improved visual cortex development secondary to correction of suboptimal nutrition. Dietary intakes of protein, zinc, calcium, and phosphorus were higher in the nutrient-enriched group compared to the control group during the 12 week feeding intervention (P<0.05). Interestingly, nutrient intakes during the feeding intervention were correlated with anthropometric measures and body composition at each baby's 4 months CA birth date. For example intakes of protein, zinc, calcium and phosphorus at 4 weeks post-discharge were positively correlated with length and head circumference (r=0.43-0.45, P<0.02) and bone mineral content (r=0.59-0.61, P<0.001) and negatively correlated with trunk fat-mass (r=−0.46-0.50, P<0.001) at the 4 month CA visit. Trunk fat-mass is associated with a metabolic profile that promotes insulin resistance and hyper-triglyceridemia which, in turn, are predictors of a number of chronic diseases including type II diabetes (53-56). While feeding intervention may not prevent chronic disease in adulthood, the direction of the aforementioned relationship suggests it won't promote it either.

General Developmental Level

The Bayley Scales of Infant Development-Second Edition (BSID-II, Psychological Corporation, San Antonio, Tex.) was administered by one of two qualified psychometrists, blinded to feeding assignment, at 18 mo CA (±10 days) (25). This examination consists of (1) the Mental Scale (MDI) which includes items that assess memory, problem solving, discrimination, classification, language and social skills; (2) the Motor Scale (PDI) which assesses control of gross and fine motor muscle groups, including walking, running, use of writing implements, and imitation of hand movements and; (3) the Behavior Rating Scale (BRS) which assesses qualitative aspects of the child's test taking behavior including orientation and engagement toward tasks and emotional regulation.

No statistically significant differences were found between feeding groups in the MDI, PDI or BRS scores assessed at 18 mo CA (Table 4). Two infants in each feeding group had an MDI score indicative of significantly delayed performance (<70), and three infants in the control and one in the intervention had an MDI score consistent with mildly delayed performance (70-84). One infant in the control group had a PDI score indicative of significant delay; whereas two infants in the control and one in the intervention had a PDI consistent with mildly delayed performance. There was a trend toward infants in the intervention group to have greater number of successfully completed tasks in the language (P=0.053) and motor (P=0.067) facets than in the control group.

TABLE 4
Developmental scores of predominantly
human milk fed preterm infants¹

| | Control | Intervention | Normal |
|---|---|---|---|
| | (n = 15) | (n = 12) | Limits |
| Mental Development | 91 (77, 107) | 100 (72, 102.5) | 84-114 |
| Index (MDI) | | | |
| Psychomotor | 94 (86, 103) | 94 (90, 99) | 84-114 |
| Development | | | |
| Index (PDI) | | | |

Facet Scores

| | | | |
|---|---|---|---|
| Cognitive | 13 (10, 15) | 14.5 (12.0, 16.5) | |
| Language² | 7 (2, 12) | 10.5 (9.0, 12.0) | |
| Social | 8 (6, 10) | 8.5 (7.0, 10.0) | |
| Motor³ | 11 (8, 13) | 12.0 (11.5, 12.5) | |
| Behaviour | 105 (100, 110) | 105 (102, 128) | 94-102 |
| Rating Scale | | | |

¹Values are the medians (1^{st}, 3^{rd} quartile). Differences between feeding groups was assessed by analysis of co-variance (parametric data) controlling for sex and birth weight stratum (≦1250 g, >1250) or Wilcoxon rank-sum test (nonparametric data). Infants (2 control, 1 intervention) with MDI scores <50 were coded as 50 for the purposes of statistical analysis and are summarized as such here.
²P = 0.053
³P = 0.067

Visual acuity and contrast sensitivity measures at 4 mo CA were not correlated with the MDI, PDI or BRS at 18 mo; however several associations were found between visual outcomes and the number of correct items achieved on the cognitive, language and social facets. Visual acuity at 4 mo was associated with the number of correct items achieved on the cognitive facet (r=0.40, P=0.04). Likewise, contrast sensitivity at the lower temporal frequency was associated with the number of correct items achieved on the cognitive (r=0.38, P=0.05) and language (rₛ=0.38, P=0.05) facets. Contrast sensitivity at the higher temporal frequency was associated with scores in the social facet (r=0.39, P=0.04). Visual acuity at 6 mo was associated with BRS scores at 18 mo (rₛ=0.48, P=0.02). Contrast sensitivity at 6 Hz at 6 mo was associated with scores on the MDI (rₛ=0.38, P=0.06), BRS (rₛ=0.40, P=0.06), and the number of correct items achieved on the cognitive (r=0.33, P=0.09) and motor (rₛ=0.38, P=0.05) facets.

Growth

Weight, length and head circumference at birth were obtained from hospital medical records. Weight, length and head circumference of infants on Study Day 1, and at 4, 8 and 12 weeks following discharge and at each infant's 3, 4, 6 and 12 mo CA birth date were examined. Growth rate (weight [g/kg/d], length [cm/wk], and head circumference [cm/wk]) of infants between Study Day 1 and 12 wk following discharge were also examined. Further, body composition (fat-free mass, fat mass, whole body bone mineral content) at 4 and 12 months CA was examined. Infants were weighed in the nude at each visit using a precision scale (±2 g, Medela BabyWeigh, Medela, Mississauga, ON; or ±10 g, MBS 2010 Baby Scale, MyWeigh, at 12 mo CA), and length was measured to the nearest 0.1 cm with a length board (Ellard Instrumentation, Munroe, Wash.). Head circumference was measured using a non-stretchable tape measure (InserTape, Abbott Nutrition, Montreal Quebec). Weight-for-age, length-for-age, and head circumference-for-age z-scores were computed at Study Day 1 using the Fenton preterm growth charts, and thereafter using WHO Anthro 2005 downloadable software and macros (27-29). Total body bone mineral content, bone mineral density, fat mass, and lean mass were measured using dual energy x-ray absorptiometry (GE Lunar Prodigy, Buckinghamshire, UK) at 4 (7 days) and 12 (10 days) months CA.

The change in weight-for-age, but not length-for-age or head circumference-for-age z-scores, during the feeding intervention (Study Day 1 to 12 weeks post-discharge) were associated with visual acuity at 4 mo CA (r=0.37, P=0.04). Changes in anthropometric z-scores during the feeding intervention were not associated with latter measures of contrast sensitivity at either 6 or 10 Hz. Infants were, on average, 2.5+0.6 and 2.3+0.43 mo CA in the control and intervention groups, respectively, at the end of the 12 week feeding intervention.

The mean z-scores from hospital discharge (Study Day 1) to the end of the 1^{st} year of life are summarized in Table 5. Infants in the intervention group had great length-for-age z scores during the first year of life (P=0.03) and tended to have greater weight-for-age z-scores (P=0.06). Unlike weight and length, a statistical interaction was found between feeding group and birth weight for head circumference-for-age z-scores (P=0.06). Infants born ≦1250 g in the intervention group had a significantly higher mean head circumference-for-age z-score than those in the control group during the first year of life (P=0.04). No significant difference in mean head circumference-for-age was observed between feeding groups among the infants born >1250 g. Re-analysis of the same growth data, including SGA infants did not change these findings. The intervention did result in infants having more bone but after correcting for the length of infants the difference between feeding groups disappeared. This likely means that the intervention supported a proportional increase in whole body bone mineral content. Importantly, it was found that the intervention also resulted in proportional increases in lean and fat mass gains.

TABLE 5
Weight, length and head circumference-for-age z-scored of predominantly
human milk-fed preterm infants born appropriate-for-gestational age^{1,2}

| Age and | | | | |
|---|---|---|---|---|
| feeding group | N | Weight³ | Length⁴ | Head Circumference⁵ |
| Discharge | | | | |
| (Study Day1) | | | | |
| Control | 15 | −0.94 ± 0.83 | −1.12 ± 0.64 | −0.11 ± 0.90 |
| Intervention | 15 | −0.64 ± 0.70 | −0.93 ± 0.73 | 0.36 ± 0.47 |
| 4 mo | | | | |
| Control | 15 | −0.88 ± 1.41 | −0.77 ± 1.22 | 0.24 ± 1.22 |
| Intervention | 15 | −0.30 ± 0.87 | 0.08 ± 1.08 | 0.83 ± 0.64 |
| 6 mo | | | | |
| Control | 15 | −0.84 ± 1.33 | −0.88 ± 1.13 | 0.14 ± 1.15 |
| Intervention | 14 | −0.25 ± 0.90 | 0.16 ± 0.99 | 0.96 ± 0.64 |
| 12 mo | | | | |
| Control | 14 | −0.52 ± 1.17 | −0.73 ± 1.40 | 0.50 ± 0.82 |
| Intervention | 12 | 0.36 ± 0.87 | 0.67 ± 0.90 | 0.93 ± 0.67 |

¹Values are the unadjusted means ± SD. Growth data were normalized to produce z-scores using the Fenton Preterm Growth Chart (28, 29) for Study Day 1 and the WHO Growth Standards (27) thereafter.
²Differences between feeding groups were assessed by repeated measures analysis of variance controlling for gender and birth weight stratum (≦1250 g, >1250 g)
³Feeding Group Main Effect, P = 0.06; Time P < 0.0001
⁴Feeding Group Main Effect, P = 0.03; Time P < 0.0001
⁵Feeding Group x birth weight stratum interaction (P = 0.06); Among infants <1250 g, Intervention > Control (P = 0.04)

It should be noted that the values of nutrients per 100 mL of a typical formulation may vary slightly due to displacement of the volume once fortifier is added to the human milk. Thus, the concentrations of the nutrients in Table 6 are intended to illustrate the ideal amounts of nutrients per 100 mL of the formulation volume.

TABLE 6
Energy and Select Nutrient Composition of Mature Human Milk
(HM) and Mature Human Milk with Multi-Nutrient Fortifier of
the present invention vs NeoSure and EnfaCare per Volume

| Energy or | A: HM Alone | B: HM + Fortifier | NeoSure | EnfaCare |
|---|---|---|---|---|
| Nutrient | (per 100 ml) | (per 100 ml) | (per 100 ml) | (per 100 ml) |
| Energy (kcal) | 65 | 80.3 | 75 | 74 |
| Protein (g) | 1.2 | 2.7 | 2.1 | 2.1 |
| (hydrolyzed whey) | | | | |
| Fat (g) | 4.0 | 4.4 | 4.1 | 3.9 |
| Carbohydrate (g) | 7.4 | 8.9 | 7.5 | 7.7 |
| Minerals | | | | |
| Calcium (mg) | 26.4 | 138.8 | 78.4 | 89 |
| Phosphorus (mg) | 12.4 | 69.4 | 46.3 | 66 |
| Magnesium (mg) | 3.4 | 9.7 | 6.7 | 8 |
| Sodium (mg) | 16.0 | 37 | 24.6 | 26 |
| Potassium (mg) | 53.4 | 110 | 106 | 78 |
| Chloride (mg) | 42.6 | 55 | 56 | 58 |
| Zinc (mg) | 0.27 | 1.2 | 0.9 | 1.25 |
| Copper (mg) | 0.06 | 0.08 | 0.09 | 0.09 |
| Iron (mg) | 0.1 | 2 | 1.3 | 1.8 |
| Manganese (ug) | 0.35 | 1 | 7.4 | 11.3 |
| Selenium (ug) | 1.8 | 4 | 1.7 | 2 |
| Vitamins | | | | |
| Vitamin A (ug) | 48.5 | 100 | 103.1 | 99.1 |
| Vitamin D (IU) | 26 | 150 | 52.2 | 59 |
| Vitamin E (mg)** | 0.5 | 1.5 | 1.8 | 2 |
| Folic Acid (ug)*** | 8.5 | 30 | 18.7 | 20 |
| Thiamin (mg) | 0.021 | 0.07 | 0.16 | 0.1 |
| Riboflavin (mg) | 0.035 | 0.1 | 0.11 | 0.1 |
| Niacin (mg) | 0.18 | 0.8 | 1.5 | 1.5 |
| Biotin (ug) | 0.6 | 1.7 | 6.7 | 4.5 |
| Vitamin B6 (mg) | 0.013 | 0.0405 | 0.07 | 0.07 |
| Vitamin B12 (ug) | 0.043 | 0.14 | 0.3 | 0.2 |
| Vitamin K (ug) | 0.25 | 0.7 | 0.82 | 6 |
| Vitamin C (mg) | 5 | 27.5 | 11.2 | 12 |
| Pantothenic Acid (mg) | 0.22 | 0.55 | 0.6 | 0.6 |
| Choline (mg) | 18.5 | 22.5 | 12 | 18 |

*Milk Composition values were obtained from the Institute of Medicine, Dietary Reference Intake Series (1997-2004). The series can be assessed at: http://www.iom.edu/CMS/3788/4574/45127.aspx
**Vit E may be added as all natural d-alpha-tocopheryl acetate
***Folate may be added as 5-methyltetrahydrofolate

TABLE 7
Energy and Select Nutrient Composition of Mature Human Milk (HM) and Mature Human Milk
with Multi-Nutrient Fortifier on an Intake Basis; based on Table 6 columns A and B

| | | | B Maximum | | |
|---|---|---|---|---|---|
| | B Avg Intake* | | Intake* | | PRNI |
| | (per kg) | Avg A + B | (per kg) | Max A + B | (per kg or d) |
| Energy or Nutrient | | | | | |
| Energy (kcal) | 41.0 | 93.0 | 69.9 | 121.9 | 100-120 |
| Protein (g) | | | | | |
| (hydrolyzed whey) | 1.4 | 2.3 | 2.3 | 3.3 | 2.2 |
| Fat (g) | 2.2 | 5.4 | 3.8 | 7.0 | 4.4-7.3 |
| Carbohydrate (g) | 4.5 | 10.5 | 7.7 | 13.7 | 7.5-15.5 |
| Minerals | | | | | |
| Calcium (mg) | 70.8 | 91.9 | 120.8 | 141.9 | 257/d |
| Phosphorus (mg) | 35.4 | 45.3 | 60.4 | 70.3 | 105/d |
| Magnesium (mg) | 4.9 | 7.7 | 8.4 | 11.2 | 4.86-14.58 |
| Sodium (mg) | 18.9 | 31.7 | 32.2 | 45.0 | 46-69 |
| Potassium (mg) | 56.1 | 98.8 | 95.7 | 138.4 | 97.8-136.9 |
| Chloride (mg) | 28.1 | 62.1 | 47.9 | 81.9 | 70.8-106.2 |
| Zinc (mg) | 0.6 | 0.8 | 1.0 | 1.3 | 0.98 |
| Copper (mg) | 0.04 | 0.1 | 0.1 | 0.1 | 0.07-0.12 |
| Iron (mg) | 1.0 | 1.1 | 1.7 | 1.8 | 2 to 4 |
| Choline (mg) | 11.5 | 26.3 | 19.6 | 34.4 | NA |
| Selenium (ug) | 1.1 | 2.6 | 1.9 | 3.4 | 3.2-4.7 |
| Vitamins | | | | | |
| Vitamin A (ug) | 51.0 | 89.8 | 87.0 | 125.8 | 400/d |
| Vitamin D (IU) | 76.5 | 97.3 | 130.5 | 151.3 | 400/d |
| Vitamin E (mg)** | 0.8 | 1.2 | 1.3 | 1.7 | 0.5 |
| Folic Acid (ug)*** | 15.3 | 22.1 | 26.1 | 32.9 | 25/d |
| Thiamin (mg) | 0.04 | 0.05 | 0.06 | 0.08 | 0.05 |
| Riboflavin (mg) | 0.05 | 0.08 | 0.09 | 0.12 | 0.05 |
| Niacin (mg) | 0.41 | 0.55 | 0.70 | 0.84 | 8.6 |
| Biotin (ug) | 0.87 | 1.35 | 1.48 | 1.96 | 1.5 |
| Vitamin B6 (mg) | 0.02 | 0.03 | 0.04 | 0.05 | 5 mg/g pro |
| Vitamin B12 (ug) | 0.07 | 0.11 | 0.12 | 0.16 | 0.15/d |
| Vitamin K (ug) | 0.36 | 0.56 | 0.61 | 0.81 | |
| Vitamin C (mg) | 14.03 | 18.03 | 23.93 | 27.93 | 20 |
| Pantothenic Acid (mg) | 0.28 | 0.46 | 0.48 | 0.65 | 0.8-1.3 |

*B Average or Maximum Intake of fortified human milk based on 4 week volumes reported in the original study. Maximum = 2 SD above mean
**Vit E may be added as all natural d-alpha-tocopheryl acetate
***Folate may be added as 5-methyltetrahydrofolate

Duration and Exclusivity of Breastfeeding. The feeding intervention had little if any affect on human milk feeding. During the feeding intervention no differences were seen between groups with respect to the number of infants being human milk-fed, the total volume of human milk provided each day or the percentage of daily feedings provided as human milk. At 12 weeks post-discharge, 71+38 and 88+15.4 percent of daily feedings in the control and +nutrients group, respectively were provided as human milk.

It is important to note that the intervention study did not negatively influence breastfeeding. This is an important observation because the length of breastfeeding is associated with latter cognitive development even after controlling for confounding variables such as maternal intelligence and quality of the home environment (31). The percentage of infants who were still being fed human milk 12 weeks postdischarge in the present study is much higher than that reported in the literature and is likely due to the fact that they were predominantly human milk-fed at discharge and the significant amount of lactation support they received at home (70-73).

The above-described embodiments of the invention are intended to be examples only. Alterations, modifications and variations can be effected to the particular embodiments by those of skill in the art without departing from the scope of the invention, which is defined solely by the claims appended hereto.

### REFERENCES

The following references are incorporated herein by reference.
1. Aggett P J, Agostoni C, Axelsson I, et al. Feeding Preterm Infants After Hospital Discharge: A Commentary by the ESPGHAN Committee on Nutrition. J Pediatr Gastroenterol Nutr 2006; 42:596-603.
2. Carlson S E. Feeding After Discharge: Growth, development and long-term effects. In: Tsang R C, Uauy R, Koletzko B, Zlotkin S H, eds. Nutrition of the Preterm Infant. Scientific Basis and Practical Guidelines. Second Edition. Cincinnati, Ohio: Digital Educational Publishing, Inc, 2005:357-81.
3. Greer F R. Feeding the Preterm Infant After Hospital Discharge. Pediatr Ann 2001; 30:658-65.
4. Griffin I J. Postdischarge nutrition for high risk neonates. Clin Perinatol 2002; 29:327-44.
5. Kleinman R. American Academy of Pediatrics. Pediatric Nutrition Handbook. 5th Edition. Elk Grove Village, Ill.: American Academy of Pediatrics, 2004.
6. O′Connor D L, Merko S, Brennan J. Human Milk Feeding of Very Low Birth Weight Infants During Initial Hospitalization and After Discharge. Nutrition Today 2004; 39:102-11.
7. Schanler R J. Post-discharge nutrition for the preterm infant. Acta Paediatr Suppl 2005; 94:68-73.
8. Bishop N J, King F J, Lucas A. Increased bone mineral content of preterm infants fed with a nutrient enriched formula after discharge from hospital. Arch Dis Child 1993; 68:573-8.
9. Lucas A. Long-term programming effects of early nutrition—implications for the preterm infant. J Perinatol 2005; 25 Suppl 2:S2-6.
10. Lucas A, Morley R, Cole T J. Randomised trial of early diet in preterm babies and later intelligence quotient. BMJ 1998; 317:1481-7.
11. Lucas A, Morley R, Cole T J, et al. Early diet in preterm babies and developmental status at 18 months. Lancet 1990; 335:1477-81.
12. Nutrition Committee of the Canadian Paediatric Society. Nutrient needs and feeding of premature infants. Can Med Assoc J 1995; 152:1765-85.
13. Uauy R, Tsang R C, Koletzko B, Zlotkin SH. Concepts, definitions and approaches to define the nutritional needs of LBW infants. In: Tsang R C, Uauy R, Koletzko B, Zlotkin SH, eds. Nutrition of the Preterm Infant. Scientific Basis and Practical Guidlines. Second Edition. Cincinnati, Ohio: Digital Educational Publishing, Inc, 2005:1-21.
14. Georgieff M K, Rao R. The role of nutrition in cognitive development. In: Nelson C A, Luciana M, eds. Handbook of Developmental Cognitive Neuroscience. London, England: The MIT Press, 2001:491-504.
15. Levitsky D A, Strupp B J. Malnutrition and the brain: changing concepts, changing concerns. J Nutr 1995; 125:2212 S-20S.
16. Kramer M S, Demissie K, Yang H, Platt R W, Sauve R, Liston R. The contribution of mild and moderate preterm birth to infant mortality. Fetal and Infant Health Study Group of the Canadian Perinatal Surveillance System. JAMA 2000; 284:843-9.
17. Statistics Canada. Births and birth rate, by province and territory. Version current 29 Sep. 2005. http://www40.statcan.ca/101/cst01/demo04b.htm.
18. Koo W W, Hockman E M. Posthospital discharge feeding for preterm infants: effects of standard compared with enriched milk formula on growth, bone mass, and body composition. Am J Clin Nutr 2006; 84:1357-64.
19. Abrams S A, Schanler R J, Tsang R C, Garza C. Bone mineralization in former very low birth weight infants fed either human milk or commercial formula: one-year follow-up observation. J Pediatr 1989; 114:1041-4.
20. Hack M, Breslau N. Very low birth weight infants: effects of brain growth during infancy on intelligence quotient at 3 years of age. Pediatrics 1986; 77:196-202.
21. Hack M, Breslau N, Weissman B, Aram D, Klein N, Borawski E. Effect of very low birth weight and subnormal head size on cognitive abilities at school age. N Engl J Med 1991; 325:231-7.
22. Latal-Hajnal B, von Siebenthal K, Kovari H, Bucher H U, Largo R H. Postnatal growth in VLBW infants: significant association with neurodevelopmental outcome. J Pediatr 2003; 143:163-70.
23. Hammoudi D S, Lee S S, Madison A, et al. Reduced visual function associated with infantile spasms in children on vigabatrin therapy. Invest Opthalmol Vis Sci 2005; 46:514-20.
24. Mirabella G, Morong S, Buncic JR, et al. Contrast sensitivity is reduced in children with infantile spasms. Invest Opthalmol Vis Sci 2007; 48:3610-5.
25. Bayley N. Bayley scales of infant development. San Antonio, Tex.: Psychological Corp, 1993.
26. Gibson R. Principles of nutritional assessment. New York: Oxford University Press, 2005.
27. World Health Organization. WHO Anthro 2005, Beta version Feb. 17, 2006: Software for assessing growth and development of the world's children. Available at: http://www.who.int/childgrowth/software/en/
28. Fenton T R. A new growth chart for preterm babies: Babson and Benda's chart updated with recent data and a new format. BMC Pediatr 2003; 3:13.
29. Fenton T R, Sauve RS. Using the LMS method to calculate z-scores for the Fenton preterm infant growth chart. Eur J Clin Nutr 2007; 61:1380-5.
30. Canadian Institute of Health Research, Natural Sciences and Engineering Research Council of Canada, Social Sciences and Humanities Research Council of Canada. Tri-Council policy statement: Ethical Conduct for Research Involving Humans. Ottawa, Ontario: Public Works and Government Services; 1998.
31. Mayer D L, Dobson V. Grating acuity cards: Validity and Reliability in Studies of Human Visual Development. In: Dobbing J, ed. Developing Brain and Behaviour: The Role of Lipids in Infant Formula. San Diego: Academic Press, 1997:254-288.
32. Bayley N. Bayley scales of infant development. San Antonio, Tex.: Psychological Corp, 1993.
33. Carver J D, Wu P Y, Hall R T, et al. Growth of preterm infants fed nutrient-enriched or term formula after hospital discharge. Pediatrics 2001; 107:683-9.
34. Chan G M. Growth and bone mineral status of discharged very low birth weight infants fed different formulas or human milk. J Pediatr 1993; 123:439-43.
35. Cooke R J, Griffin I J, McCormick K, et al. Feeding preterm infants after hospital discharge: effect of dietary manipulation on nutrient intake and growth. Pediatr Res 1998; 43:355-60.
36. Lapillonne A, Salle B L, Glorieux F H, Claris O. Bone mineralization and growth are enhanced in preterm infants fed an isocaloric, nutrient-enriched preterm formula through term. Am J Clin Nutr 2004; 80:1595-603.
37. Lucas A, Bishop N J, King F J, Cole T J. Randomised trial of nutrition for preterm infants after discharge. Arch Dis Child 1992; 67:324-7.
38. Wheeler R E, Hall R T. Feeding of premature infant formula after hospital discharge of infants weighing less than 1800 grams at birth. J Perinatol 1996; 16:111-16.
39. Health Canada. Exclusive breastfeeding duration. 2004. http://www.hc-sc.gc.ca/fn-an/nutrition/child-enfant/infant-nourisson/excl_bf_dur-dur_am_excl_e.html.
40. World Health Organization. Global Strategies for Infant and Young Child Feeding. Resolution Passes at Fifty-fourth World Health Assembly; May 9, 2001.
41. Canadian Paediatric Society, Dietitians of Canada, Health Canada (CPS/DC/HC). “Nutrition for Healthy Term Infants”. Ottawa, Canada: Minister of Public Works and Government Services, 1998.
42. American Academy of Pediatrics. Breastfeeding and the use of human milk. Pediatrics 2005; 115:496-506.
43. Pinelli J, Atkinson S A, Saigal S. Randomized trial of breastfeeding support in very low-birth-weight infants. Arch Pediatr Adolesc Med 2001; 155:548-53.
44. Institute of Medicine. Vitamin E. Dietary Reference Intakes For Vitamin C, Vitamin E, Selenium and Carotenoids. Washington, D.C.: National Academy Press, 2000:186-283.
45. Atkinson S. Effects of Gestational Stage at Delivery on Human Milk Components. In: Jensen R, editor. Handbook of Milk Composition. San Diego: Academic Press; 1995. p. 222-237.
46. Klein C J. Nutrient requirements for preterm infant formulas. J Nutr 2002; 132:1395 S-577S.
47. Abrams S A, Schanler R J, Garza C. Bone mineralization in former very low birth weight infants fed either human milk or commercial formula. J Pediatr 1988; 112:956-60.
48. Cooke R J, McCormick K, Griffin I J, et al. Feeding preterm infants after hospital discharge: effect of diet on body composition. Pediatr Res 1999; 46:461-4.
49. Friel J K, Andrews W L, Matthew J D, et al. Zinc supplementation in very-low-birth-weight infants. J Pediatr Gastroenterol Nutr 1993; 17:97-104.
50. Hall R T, Wheeler RE, Rippetoe L E. Calcium and phosphorus supplementation after initial hospital discharge in breast-fed infants of less than 1800 grams birth weight. J Perinatol 1993; 13:272-8.
51. Schanler R J, Abrams S A. Postnatal attainment of intrauterine macromineral accretion rates in low birth weight infants fed fortified human milk. J Pediatr 1995; 126:441-7.
52. Wauben I P, Atkinson SA, Shah J K, Paes B. Growth and body composition of preterm infants: influence of nutrient fortification of mother's milk in hospital and breastfeeding post-hospital discharge. Acta Paediatr 1998; 87:780-5.
53. Bjorntorp P. Metabolic implications of body fat distribution. Diabetes Care 1991; 14:1132-43.
54. de Vegt F, Dekker J M, Jager A, et al. Relation of impaired fasting and postload glucose with incident type 2 diabetes in a Dutch population: The Hoorn Study. Jama 2001; 285:2109-13.
55. Esmaillzadeh A, Mirmiran P, Azizi F. Clustering of metabolic abnormalities in adolescents with the hypertriglyceridemic waist phenotype. Am J Clin Nutr 2006; 83:36-46; quiz 183-4.
56. Kelley D E, Williams K V, Price J C, McKolanis T M, Goodpaster B H, Thaete F L. Plasma fatty acids, adiposity, and variance of skeletal muscle insulin resistance in type 2 diabetes mellitus. J Clin Endocrinol Metab 2001; 86:5412-9.
57. Niday Perinatal Database for the Greater Toronto Area. First Annual Statistical Report 2003/2004. http://www.childhealthnetwork.com/chn/pdfs/Niday%20Perinatal%20-%20Fact%20Sheet%205.pdf.
58. Gibson R. Principles of Nutritional Assessment. 2nd edition. New York: Oxford University Press, 2005.
59. Brunton J A, Bayley H S, Atkinson S A. Validation and application of dual-energy x-ray absorptiometry to measure bone mass and body composition in small infants. Am J Clin Nutr 1993; 58:839-45.
60. Brunton J A, Bayley H S, Atkinson S A. Body composition analysis by dual energy X-ray absorptiometry compared to chemical analysis of fat, lean and bone mass in small piglets. Basic Life Sci 1993; 60:157-60.
61. Koo W W, Massom L R, Walters J. Validation of accuracy and precision of dual energy X-ray absorptiometry for infants. J Bone Miner Res 1995; 10:1111-5.
62. Picaud J C, Rigo J, Nyamugabo K, Milet J, Senterre J. Evaluation of dual-energy X-ray absorptiometry for body-composition assessment in piglets and term human neonates. Am J Clin Nutr 1996; 63:157-63.
63. Health Canada, Canadian Nutrient File 2005. http://www.hc-sc.gc.ca/fn-an/nutrition/fiche-nutri-data/index_e.html.
64. Wainwright P E, Colombo J. Nutrition and the development of cognitive functions: interpretation of behavioral studies in animals and human infants. Am J Clin Nutr 2006; 84:961-70.
65. McCall R B, Mash C W. Long-chain polyunsaturated fatty acids and the measurement and prediction of intelligence. In: Dobbing J, ed. Developing Brain and Behaviour: The role of lipids in infant nutrition. San Diego: Academic Press, 1997:295-338.
66. Columbo J. Individual differences in infant cognition: methods, measures and models. In: Dobbing J, ed. Developing Brain and Behaviour: The role of lipids in infant nutrition. San Diego: Academic Press, 1997:339-385.
67. Bayley N. Bayley Scales of Infant Development. San Antonio, Tex.: Psychological Corp., 1993.
68. Hosmer D W, S. L. Model building strategies and methods for logistic regression. New York: John Wiley & Sons Inc, 1989.
69. O′Connor DL, Khan S, Weishuhn K, Vaughan J, Jefferies A, Campbell DM, Asztalos E, Feldman M, Rovet J, Westall C, Whyte H. Growth and Nutrient Intakes of Human Milk-Fed Premature Infants Provided with Extra Energy and Nutrients after Hospital Discharge: A Pilot Randomized Controlled Trial. Pediatrics 121 (4) (in press).
70. Callen J, Pinelli J. A review of the literature examining the benefits and challenges, incidence and duration, and barriers to breastfeeding in preterm infants. Adv Neonatal Care. 2005; 5(2):72-88; quiz 89-92.
71. Furman L, Minich N, Hack M. Correlates of lactation in mothers of very low birth weight infants. Pediatrics. 2002; 109(4):e57.
72. Kaufman K J, Hall L A. Influences of the social network on choice and duration of breast-feeding in mothers of preterm infants. Res Nurs Health. 1989; 12(3):149-159.
73. Pinelli J, Atkinson S A, Saigal S. Randomized trial of breastfeeding support in very low-birth-weight infants. Arch Pediatr Adolesc Med. 2001; 155(5):548-553.
74. Weishuhn K, Westall C, Khan S, Vaughan J, Jefferies A, Campbell D M, Asztalos E, Feldman M, Rovet J, Westall C, Whyte H, O′Connor D L. Visual development of premature infants fed human milk containing extra energy and nutrients after hospital discharge. E-PAS2007:61, 5740.4, 2007.

1. A multi-nutrient fortifier in the form of a concentrated liquid, powder, granules, or paste for supplementing human breast milk for delivery to an infant, wherein the infant is a low birth weight infant, a post-surgical infant, or an ill term-born infant.

2. (canceled)

3. (canceled)

4. The fortifier of claim 1, comprising about 44% or more protein, by weight, of energy-contributing ingredients in the fortifier.

5. The fortifier of claim 1, comprising about 17% protein, by weight of energy-contributing ingredients, when combined with human breast milk.

6. The fortifier of claim 1 which, in combination with human breast milk, forms a formulation comprising, per 100 mL total volume, the following nutrients:
a) energy: 81±about 20% kcal;
b) 2.7 g±about 20% hydrolyzed whey protein;
c) 4.4 g±about 20% of fat;
d) 8.9 g±about 20% of carbohydrate;
e) 138.8 mg±about 20% of calcium;
f) 69.4 mg±about 20% of phosphorus;
g) 9.7 mg±about 20% of magnesium;
h) 37 mg±about 20% of sodium;
i) 110 mg±about 20% of potassium;
j) 55 mg±about 20% of chloride;
k) 1.2 mg±about 20% of zinc;
l) 0.08 mg±about 20% of copper;
m) 2.0 mg±about 20% of iron;
n) 4 μg±about 20% of selenium;
o) 100 μg±about 20% of Vitamin A;
p) 150 IU±about 20% of Vitamin D;
q) 1.5 mg±about 20% of Vitamin E;
r) 30 μg±about 20% of folic acid;
s) 1.0 μg±about 20% of manganese;
t) 0.07 mg±about 20% of thiamin;
u) 0.1 mg±about 20% of riboflavin;
v) 0.8 mg±about 20% of niacin;
w) 1.7 μg±about 20% of biotin;
x) 0.0405 mg±about 20% of Vitamin B6;
y) 0.14±about 20% of Vitamin B12;
z) 0.7 μg±about 20% of Vitamin K;
aa) 27.5 mg±about 20% of Vitamin C;
bb) 0.55 mg±about 20% of pantothenic acid; and
cc) 22.5 mg±about 20% of choline.

7. The fortifier of claim 1, wherein the fortifier is sterile.

8. The fortifier of claim 1, wherein the fortifier is formulated for delivery after hospital discharge.

9. A method of improving growth and body composition of a breastfed infant, wherein the infant is a low birth weight infant, a post-surgical infant, or an ill term-born infant, comprising:
a) supplementing human breast milk with a multi-nutrient fortifier to produce a supplemented breast milk formulation, said fortifier being in the form of a concentrated liquid, powder, granules, or paste; and
b) administering to the infant the supplemented breast milk formulation, thereby improving growth and body composition of the infant.

10. (canceled)

11. (canceled)

12. The method of claim 9, wherein the fortifier comprises about 44% or more protein, by weight, of energy-contributing ingredients in the fortifier.

13. The method of claim 9, wherein the fortifier comprises about 17% protein, by weight of energy-contributing ingredients, when combined with human breast milk.

14. The method of claim 9, wherein the fortifier, in combination with human breast milk, forms a formulation comprising, per 100 mL total volume, the following nutrients:
a) energy: 81±about 20% kcal;
b) 2.7 g±about 20% hydrolyzed whey protein;
c) 4.4 g±about 20% of fat;
d) 8.9 g±about 20% of carbohydrate;
e) 138.8 mg±about 20% of calcium;
f) 69.4 mg±about 20% of phosphorus;
g) 9.7 mg±about 20% of magnesium;
h) 37 mg±about 20% of sodium;
i) 110 mg±about 20% of potassium;
j) 55 mg±about 20% of chloride;
k) 1.2 mg±about 20% of zinc;
l) 0.08 mg±about 20% of copper;
m) 2.0 mg±about 20% of iron;
n) 4 μg±about 20% of selenium;
o) 100 μg±about 20% of Vitamin A;
p) 150 IU±about 20% of Vitamin D;
q) 1.5 mg±about 20% of Vitamin E;
r) 30 μg±about 20% of folic acid;
s) 1.0 μg±about 20% of manganese;
t) 0.07 mg±about 20% of thiamin;
u) 0.1 mg±about 20% of riboflavin;
v) 0.8 mg±about 20% of niacin;
w) 1.7 μg±about 20% of biotin;
x) 0.0405 mg±about 20% of Vitamin B6;
y) 0.14±about 20% of Vitamin B12;
z) 0.7 μg±about 20% of Vitamin K;
aa) 27.5 mg±about 20% of Vitamin C;
bb) 0.55 mg±about 20% of pantothenic acid; and
cc) 22.5 mg±about 20% of choline.

15. The method of claim 9, wherein the fortifier is sterile.

16. The method of claim 9, wherein the growth and body composition of the infant are improved post hospital discharge.

17. (canceled)

18. (canceled)

19. (canceled)

20. (canceled)

21. (canceled)

22. A method of improving brain development in an infant, the infant being a low birth weight infant, a post-surgical infant, or an ill term-born infant, comprising:
supplementing human breast milk with a multi-nutrient fortifier in the form of a concentrated liquid, powder, granules, or paste, to produce a supplemented breast milk formulation, the fortifier comprising about 44% or more protein, by weight, of energy-contributing ingredients in the fortifier; and
providing the infant with the supplemented breast milk formulation after hospital discharge.

23. The method of claim, 22 wherein improving brain development comprises increasing visual acuity, increasing language and motor skills development, or increasing head circumference.

24. The method of claim 22, wherein the supplemented breast milk formulation comprises, per 100 mL total volume, the following nutrients:
a) energy: 81±about 20% kcal;
b) 2.7 g±about 20% hydrolyzed whey protein;
c) 4.4 g±about 20% of fat;
d) 8.9 g±about 20% of carbohydrate;
e) 138.8 mg±about 20% of calcium;
f) 69.4 mg±about 20% of phosphorus;
g) 9.7 mg±about 20% of magnesium;
h) 37 mg±about 20% of sodium;
i) 110 mg±about 20% of potassium;
j) 55 mg±about 20% of chloride;
k) 1.2 mg±about 20% of zinc;
l) 0.08 mg±about 20% of copper;
m) 2.0 mg±about 20% of iron;
n) 4 μg±about 20% of selenium;
o) 100 μg±about 20% of Vitamin A;
p) 150 IU±about 20% of Vitamin D;
q) 1.5 mg±about 20% of Vitamin E;
r) 30 μg±about 20% of folic acid;
s) 1.0 μg±about 20% of manganese;
t) 0.07 mg±about 20% of thiamin;
u) 0.1 mg±about 20% of riboflavin;
v) 0.8 mg±about 20% of niacin;
w) 1.7 μg±about 20% of biotin;
x) 0.0405 mg±about 20% of Vitamin B6;
y) 0.14±about 20% of Vitamin B12;
z) 0.7 μg±about 20% of Vitamin K;
aa) 27.5 mg±about 20% of Vitamin C;
bb) 0.55 mg±about 20% of pantothenic acid; and
cc) 22.5 mg±about 20% of choline.
